(19) United States
(12) Patent Application Publication
(10) Pub. No.: US 2009/0299055 A1
(43) Pub. Date: Dec. 3, 2009

(54) Purification of Sucralose Containing Feed Streams for Sucralose Crystallization

(75) Inventors: James Edwin Wiley, JR., Daphne, AL (US); John Kerr, South Croydon (GB); Robert Jansen, Portela LRS (PT); Gordon Walker, Goring Heath (GB)

Correspondence Address: Ratner Prestia, P.O. Box 980, Valley Forge, PA, 19482, US

(73) Assignee: Tate & Lyle Technology Limited, London (GB)

(21) Appl. No.: 12/412,832

(22) Filed: Mar. 27, 2009

Related U.S. Application Data
(60) Provisional application No. 61/042,122, filed on Apr. 3, 2008.

(51) Int. Cl. C07H 1/06

(52) U.S. Cl. 536/127

(57) ABSTRACT

A process for the recovery of a carbohydrate selected from sucralose and sucralose-6-esters from an aqueous feed stream is disclosed. The process comprises extracting the feed stream with an organic solvent, such as ethyl acetate, back extracting the resulting carbohydrate-containing organic extract with an aqueous solvent, and either crystallizing the carbohydrate from the organic solvent or replacing the organic solvent with water and crystallizing the carbohydrate from water. The process results in an increased overall yield of the carbohydrate.

### CROSS REFERENCE TO RELATED APPLICATIONS

This application claims priority benefit of U.S. Provisional Appln. No. 61/042,122, filed Apr. 3, 2008, the entirety of which is incorporated herein by reference.

### FIELD OF THE INVENTION

This invention relates to sucralose and to methods for its preparation. In particular, this invention relates a process for the recovery of sucralose from an aqueous sucralose containing feed stream.

### BACKGROUND OF THE INVENTION

Sucralose (4,1′,6′-trichloro-4,1′,6′-trideoxygalactosucrose), a high-intensity sweetener that can be used in many food and beverage applications, is a galacto-sucrose having the following structure:

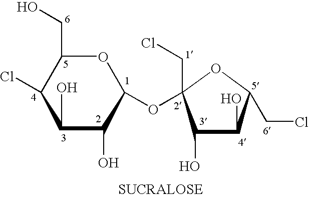

Sucralose is made from sucrose by converting the hydroxyls in the 4, 1′, and 6′ positions to chloro groups. In this process, the stereochemical configuration at the 4 position is inverted.

In one process for making sucralose from sucrose, sucrose is first converted to a sucrose-6-ester, such as sucrose-6-acetate or sucrose-6-benzoate. The sucrose-6-ester is chlorinated by reaction with a chlorination agent and a tertiary amide, and the resulting reaction mixture heated and then quenched with aqueous alkali. The resulting 4,1′,6′-trichloro-4,1′,6′-trideoxygalactosucrose ester (sucralose-6-ester) is converted to sucralose, which is subsequently purified and isolated.

This process typically provides a product that contains varying amounts of other chlorinated sugar compounds in addition to sucralose. During removal of these impurities the loss of sucralose should be minimized, and the purification and isolation process should be economical to operate on a large scale. Although advances have been made in the purification of sucralose, there is a continuing need for processes that remove impurities from sucralose, produce sucralose in high purity, minimize the yield loss in the purification process, and are economical to operate on a large scale.

### SUMMARY OF THE INVENTION

In one aspect, the invention provides a process comprising, in order, the steps of:

a) providing an aqueous feed stream comprising chlorinated saccharide impurities and a carbohydrate selected from the group consisting of sucralose, sucralose-6-esters, and mixtures thereof;

b) optionally, concentrating the aqueous feed stream;

c) extracting the aqueous feed stream with an organic solvent and producing a first organic extract and a first aqueous extract, in which the organic solvent is immiscible with water, and in which the carbohydrate is preferentially extracted into the first organic extract;

d) extracting the first organic extract with water and producing a second organic extract and a second aqueous extract, in which the carbohydrate preferentially remains in the second organic extract; and

e) crystallizing the carbohydrate from the second organic extract;

wherein the process further comprises recycling the second aqueous extract to the aqueous feed stream.

In another aspect, the invention provides a process comprising the steps of:

a) extracting an aqueous feed stream comprising sucralose, salts and chlorinated saccharide impurities with a first organic solvent and producing a first organic extract and a first aqueous extract, in which the first organic solvent is immiscible with water, and in which a portion of the sucralose passes into the first organic extract;

b) optionally, extracting the first organic extract with an aqueous solvent to produce a second organic extract and a second aqueous extract, in which the sucralose preferentially passes into the second aqueous extract, and recycling the second aqueous extract to step a);

c) optionally, concentrating the first aqueous extract;

d) extracting the first aqueous extract with a second organic solvent and producing a third organic extract and a third aqueous extract;

e) extracting the third organic extract with water and producing a fourth organic extract and a fourth aqueous extract; and

f) crystallizing the sucralose from the fourth organic extract.

Processes according to the invention increase the purity of carbohydrate-containing feed steam to the crystallizer and enhance the yield of carbohydrate.

### BRIEF DESCRIPTION OF THE DRAWINGS

FIG. 1 is flow diagram showing one process for the preparation of an aqueous sucralose containing feed stream.

FIG. 2 shows the effect of the ratio of organic solvent (“solvent”) to aqueous sucralose containing feed stream (“feed”) at constant sucralose yield on the purity of the sucralose in the first aqueous extract.

FIG. 3 is flow diagram showing a process for the treatment of a sucralose containing feed stream.

FIG. 4 is a plot showing the effect of carbohydrate concentration on the partition coefficient, K, of sucralose between an organic solvent and water.

FIG. 5 is a plot showing the effect of solvent to feed ratio in extraction EXT2 on the yield of sucralose.

FIG. 6 is a flow diagram showing the process of the invention.

FIG. 7 is a plot showing the effect of the purity of the sucralose containing organic feed stream on sucralose yield.

FIG. 8 is a plot showing the effect of the purity of the sucralose containing organic feed stream on sucralose yield.

### DETAILED DESCRIPTION OF THE INVENTION

Unless the context indicates otherwise, in the specification and claims, the terms organic solvent, first organic solvent, second organic solvent, tetrachloro saccharide, trichloro saccharide, dichloro saccharide, salt, sucralose-6-ester, carbohydrate, and similar terms also include mixtures of such materials. The term saccharide includes monosaccharide, disaccharides, and polysaccharides. Solvent means a liquid that dissolves another material. An aqueous solvent is one in which water is the primary (greater than 50 vol % of the solvents present) or only solvent. Partition coefficient, K, of a carbohydrate between an organic solvent and water is the concentration of the carbohydrate in the organic phase divided by the concentration of the carbohydrate in the aqueous phase when equal volumes of organic solvent and water are used. Two solvents are immiscible if, in any proportion, they do not form a homogeneous phase. Crystallization includes processes in which a solution is rendered saturated or supersaturated with respect to a dissolved component, and the formation of crystals of this component is achieved. The initiation of crystal formation can be spontaneous, or it may require the addition of seed crystals. Crystallization also describes the situation in which a solid or liquid material is dissolved in a solvent to yield a solution which is then rendered saturated or supersaturated so as to obtain crystals. Also, included in the term crystallization are the ancillary processes of washing the crystals with one or more solvents, drying the crystals, and harvesting the final product so obtained. Unless otherwise specified, all percentages are percentages by weight, all temperatures are in degrees Centigrade (degrees Celsius), and all solvent ratios are volume to volume.

A process for the preparation of sucralose from sucrose involves the following steps. First, the hydroxyl in the 6 position of sucrose is blocked with an ester group, such as acetate or benzoate. Then the hydroxyls in the 4, 1′, and 6′ positions of the resulting sucrose 6-ester are converted to chloro groups, with inversion of the stereochemical configuration at the 4 position. Conversion of the hydroxyls in the 4, 1′, and 6′ positions of the ester to chloro groups with inversion of the stereochemical configuration at the 4 position is disclosed in Walkup, U.S. Pat. No. 4,980,463; Jai, U.S. Pat. Pub. 2006/0205936 A1; and Fry, U.S. Pat. Pub. 2007/0100139 A1; the disclosures of which are all incorporated herein by reference. Then the ester group in the 6 position of the resulting sucralose-6-ester is removed, and sucralose, the resulting product, purified and isolated. The process, or any of the individual steps thereof, can be either batch or continuous processes.

### Preparation of Sucralose Containing Feed Streams

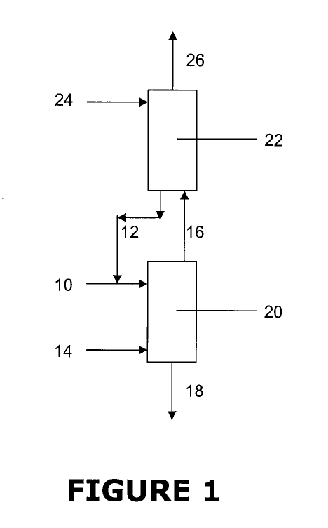

Referring to FIG. 1, following conversion of sucralose-6-ester to sucralose, an aqueous feed stream (10) that comprises sucralose is produced. Aqueous feed stream 10 typically comprises a total of about of 6 wt % to 50 wt %, for example, about 6 wt % to 12 wt %, about 12 wt % to 18 wt %, about 18 wt % to 25 wt %, or about 25 wt % to about 50 wt % of carbohydrates in a stream in which water is the primary or only solvent. Of the carbohydrates present, between 50% and 80% are typically sucralose. The other carbohydrates primarily fall into one of three categories based on the number of chlorine atoms on the molecule: tetrachloro saccharide impurities (tetrachloro saccharides), dichloro saccharide impurities (dichloro saccharides), and trichloro saccharide impurities (trichloro saccharides). The location and extent of chlorination strongly affects the polarity of the resulting saccharide. In general, the tetrachloro saccharides impurities are less polar than sucralose, and the dichloro saccharide impurities are more polar than sucralose. Generally, more polar impurities are more soluble than sucralose in more polar solvents, and less polar impurities are more soluble than sucralose in less polar solvents.

Other materials that can be present in aqueous feed stream 10 include inorganic salts, such as alkali metal chlorides such as sodium chloride, alkaline earth chlorides, and ammonium chloride; and organic salts, primarily alkali metal acetates, such as sodium acetate; dimethyl amine hydrochloride; and alkali metal formates, such as sodium formate. A small amount, typically less than 5,000 ppm, of the polar aprotic solvent used in the chlorination step, typically N,N-dimethyl formamide, can also be present in the feed stream.

Aqueous feed stream 10 and, if present, second aqueous extract 12, discussed below, are combined to produce a combined aqueous stream, which is extracted with a stream of first organic solvent (14) to produce a first organic extract (16) and a first aqueous extract (18). This extraction step is referred to as step EXT1. Because the less polar compounds are preferentially extracted into first organic extract 16, this extraction removes less polar compounds, which include the tetrachloro saccharides, as well as a portion of the sucralose from the combined aqueous stream. The extraction can be carried out under conditions in which greater than 50%, greater than 55%, greater than 60%, or greater than 65%, of the sucralose and 95% of the tetrachloro saccharide impurities in the aqueous feed stream are extracted into first organic extract 16. In an alternative embodiment, the extraction can be carried out as disclosed in WO03/076453, namely wherein a majority (i.e greater than 50%) of the tetrachlorosucrose compounds in the aqueous feed stream 10 are extracted into the first organic extract 16, and a majority (i.e. greater than 50%) of the sucralose is retained in the first aqueous extract 18.

The choice of solvent is determined by the relative solubilities of sucralose and the principal impurities in the organic solvent and in the aqueous feed stream, as well as such other factors as flammability, ease of recycling within the process, environmental concerns, toxicity, and cost. If desired, the organic solvent can be intentionally saturated with water before use in the extraction step. Mixtures of organic solvents can be used. Solvents contemplated for use as the first organic solvent include those that are immiscible with water and in which halogenated sucrose derivatives, such as sucralose, are readily soluble. Also included are solvents that are partially soluble in a first solvent such as water, an aqueous solution, or other solvent in which halogenated sucrose derivatives are readily soluble, but in which the second solvent still forms a separate phase when mixed with the first solvent in proper ratios and under proper conditions. Typical first organic solvents include, but are not limited to, methyl acetate, ethyl acetate, methyl ethyl ketone, methyl iso-butyl ketone, methyl iso-amyl ketone, methylene chloride, chloroform, diethyl ether, methyl t-butyl ether, n-pentane, n-hexane, n-heptane, n-octane, isooctane, 1,1,1-trichloroethane, n-dodecane, white spirit, turpentine, cyclohexane, propyl acetate, butyl acetate, amyl acetate, carbon tetrachloride, xylene, toluene, benzene, trichloroethylene, 2-butoxyethanol acetate (butyl CELLOSOLVE® acetate), ethylene dichloride, butanol, morpholine, and mixtures thereof. The first organic solvent preferably comprises methyl acetate, ethyl acetate, iso-propyl acetate, n-propyl acetate, n-butyl acetate, amyl acetate, methyl ethyl ketone, methyl iso-butyl ketone, methyl iso-amyl ketone, methylene chloride, chloroform, or n-butanol, either as a single solvent, or as a mixed solvent with these solvents, or with other solvents from the first list. The first solvent more preferably comprises ethyl acetate, iso-propyl acetate, n-propyl acetate, n-butyl acetate, methyl iso-butyl ketone, or n-butanol, either as a single solvent, or as a mixed solvent with these solvents, or with other solvents from the first or second list. Ethyl acetate is the most preferred solvent. Diethyl ether, methyl t-butyl ether, n-pentane, n-hexane, n-heptane, n-octane, isooctane, 1,1,1-trichloroethane, n-dodecane, white spirit, turpentine, cyclohexane, carbon tetrachloride, xylene, toluene, benzene, trichloroethylene, 2-butoxyethanol acetate (butyl CELLOSOLVE® acetate), ethylene dichloride, and morpholine are generally not preferred as single solvents, but may be used in mixed solvents as described.

Extraction is carried out in a first liquid extractor (20), which can be any type of liquid-liquid extractor known in the art, for example, a conventional mixer-settler or a bank of conventional mixer-settlers, an Oldshue-Rushton multiple-mixer column, a sieve tray column, a random packed column, a pulsed packed column, a structured (SMVP) packing column, an asymmetric rotating disk extractor (ARD), a KARR® column, a Kuhni extractor, a Treybel extractor, a Scheibel column, a rotating disc contactor (RDC) column, or a centrifugal extractor such as a Podbielniak centrifugal extractor or a Robatel centrifugal extractor. An extractor with five or more theoretical stages of extraction can be used. A first organic solvent 14, for example ethyl acetate which, if desired, can be saturated with water, is fed to the bottom of extractor 20 in proportion to the total amount of feed to the top of extractor 20.

First aqueous extract 18 comprises sucralose as well as some impurities, primarily salts and saccharide impurities that are more polar than sucralose or which have about the same polarity as sucralose. If desired, first organic extract 16 can be sent to a second liquid extractor (22) to recover sucralose from first organic extract 16 while leaving the bulk of the less polar impurities in an organic extract. This extraction step is referred to as step EXT1B. If the process comprises additional purification steps, if desired, one or more other recycle streams from these additional purification steps can be recycled to the second liquid extractor 22. Second liquid extractor 22 can be any type of liquid-liquid extractor known in the art, examples of which are listed above. An extractor with five or more theoretical stages of extraction can be used. First organic extract 16 is fed into the bottom of liquid extractor 22. A stream (24) of water, which if desired can be saturated with the same organic solvent used in first liquid extractor 20, for example, water saturated with ethyl acetate, is fed into the top of extractor 22. The mass ratio of water to first organic extract 16 is typically about 0.8 to about 0.9. An interface between the two phases is maintained in the bottom of second liquid extractor 22 where the aqueous phase, second aqueous extract 12, is collected. Second aqueous extract 12 is recycled to first liquid extractor 20. Greater than 85%, 90%, 92%, or 95% of the sucralose present in the first organic phase is extracted into the second aqueous phase by step EXT1B.

The organic extract, second organic extract 26, exits the top of extractor 22. Second organic extract 26 contains less polar impurities, such as the tetrachloro saccharides. It is purged from the process and the organic solvent recovered for reuse. If the second liquid extraction (step EXT1B) is not present in the process, the first organic extract is purged from the process, and the organic solvent recovered for reuse.

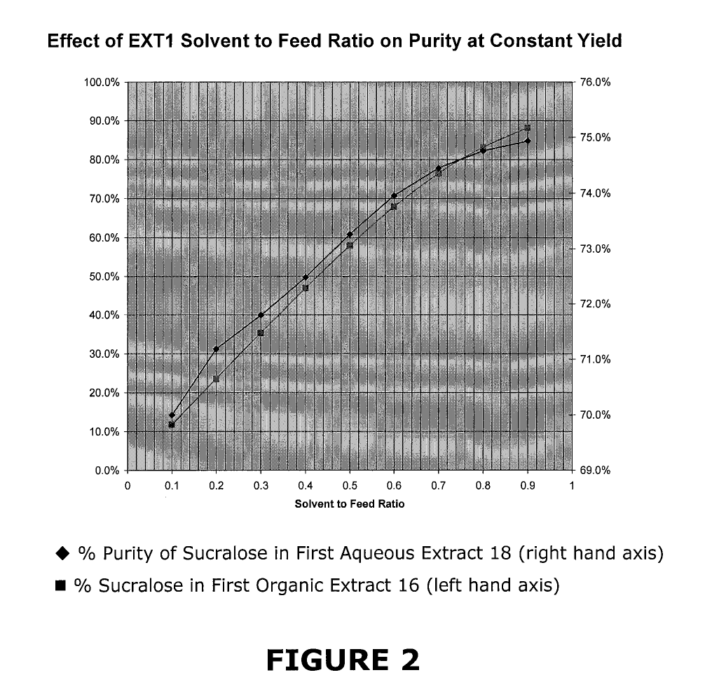

In one aspect of the invention, the mass ratio of first organic solvent 14 to aqueous feed stream 10 in the first extraction step (EXT1) is about 0.4 to about 0.9. Preferably, the mass ratio of first organic solvent 14 to aqueous feed stream 10 in step EXT1 is about 0.6 to about 0.9. FIG. 2 shows the amount of sucralose in first organic extract 16 (left hand axis) and the purity of the sucralose in first aqueous extract 18 (right hand axis) as a function of the mass ratio of first organic solvent 14 to aqueous feed stream 10 in the first extraction step (EXT1), calculated at constant sucralose yield. These values are for the process described above, a process in which a sucralose containing aqueous feed stream is extracted with an organic solvent in a first extraction step, the resulting organic extract back extracted with water in a second extraction step, and the resulting second aqueous extract recycled to the first extraction step.

As can be seen from FIG. 2, when the mass ratio of organic solvent 14 to aqueous feed stream 10 in the first extraction step is about 0.4 or greater, about 50% or more of the sucralose is extracted into first organic extract 16. When the mass ratio is 0.5 or greater, greater than about 60% of the sucralose is extracted into first organic extract 16. When the mass ratio is 0.6 or greater, greater than about 65% of the sucralose is extracted into first organic extract 16. Surprisingly, the level of impurities in first aqueous extract 18 is reduced significantly, with little or no decrease in overall sucralose yield, when higher organic solvent to aqueous feed stream ratios are used in the first extraction step.

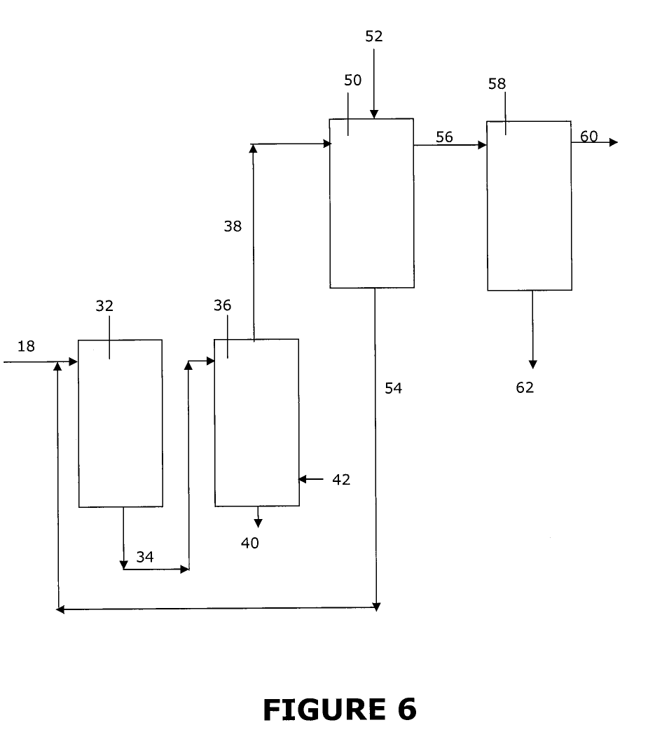

Referring to FIG. 6, in one aspect of the invention, if desired, feed stream 18 can be fed to a concentrator (32), which can be designed for either a batch or continuous operation. Concentrator 32 can be any type of evaporator known in the art, for example, a falling film evaporator, a thin-film evaporator, a wiped film evaporator, a forced circulation evaporator, a bulk evaporator, a Robert evaporator, a Herbert evaporator, a Caddle-type evaporator, or an Oskar evaporator. As described below, other sucralose containing aqueous streams can be recycled to concentrator 32.

Concentrator 32, if present, increases the concentration of carbohydrates, including the concentration of sucralose, and, if present, the salt present in sucralose containing aqueous feed stream 18. Concentrator 32 typically increases the concentration of carbohydrates in sucralose containing aqueous feed stream 18 by a factor of about 1.1 to about 4.0, or about 1.15 to about 2.5, or about 1.2 to about 2.0. Sucralose containing aqueous feed stream 18 entering concentrator 32 can have less than about 18 wt %, less than about 15 wt %, less than about 12 wt %, less than about 10 wt %, less than 9 wt %, or less than 8 wt % total carbohydrates, and more than 3 wt %, or more than 4 wt %, or more than 5 wt %, for example, about 3 wt % to about 18 wt %, 4 wt % to about 16 wt %, about 4 wt % to about 15 wt %, about 4 wt % to about 12 wt %, about 4 wt % to about 10 wt %, about 4 wt % to about 8 wt %, or about 5 wt % to about 8 wt %, total carbohydrate. Sucralose containing aqueous feed stream 18 can contain up to 18 wt % of inorganic salts, primarily alkali metal chlorides, such as sodium chloride, and organic salts, primarily alkali metal acetates, such as sodium acetate. Concentrated sucralose containing aqueous feed stream 34 leaving concentrator 32 can have at least about 10 wt %, at least about 12 wt %, at least about 13 wt %, at least 15 wt %, at least 18 wt %, at least 20 wt %, at least 22 wt %, or at least 25 wt %, and 50 wt % or less, 45 wt %, or 40 wt % or less total carbohydrate; for example, about 10 wt %, about 12 wt %, about 15 wt %, or about 18 wt % to about 25 wt %; about 10 wt %, about 12 wt %, about 15 wt %, or about 18 wt % to about 20 wt %; about 10 wt %, about 12 wt %, or about 15 wt % to about 18 wt %; about 13 wt % to about 17 wt %; about 14 wt % to about 16 wt %; or about 15 wt % to about 16 wt % total carbohydrate. Typically, sucralose comprises about 60% to 85% of the carbohydrates present in concentrated sucralose containing aqueous feed stream 34.

### Crystallization of Sucralose

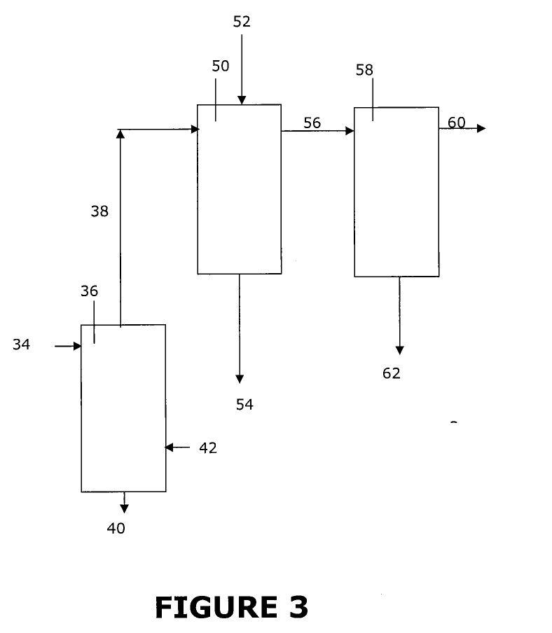

In the process of the invention, an aqueous feed stream comprising sucralose and chlorinated saccharide impurities is provided. FIG. 3 shows a process of the invention, in which the aqueous sucralose containing feed stream is extracted with an organic solvent to move the sucralose into an organic phase, the organic phase is back extracted with water, and the sucralose crystallized from the organic solvent. The aqueous feed stream can be, for example, an aqueous feed stream that has previously undergone one or more steps to remove impurities, such as first aqueous extract 18. In one aspect of the invention, the aqueous feed stream is concentrated sucralose containing aqueous stream 34. For convenience, the invention has been described as a process in which concentrated sucralose containing aqueous stream 34 is the sucralose containing aqueous feed stream. However, other sucralose containing aqueous feed streams can be used in the invention.

Feed stream 18, or, if concentrator 32 is present, concentrated aqueous stream 34 is fed to third liquid extractor 36. This extraction step is referred to as step EXT2 and can be either batch or continuous. Third liquid extractor 36 can be any type of liquid-liquid extractor known in the art, examples of which are given above. In this extraction step, sucralose is extracted into a second organic solvent (42) to form a third organic extract (38). Most of the more polar impurities and most of the salts present in the aqueous feed remain in the third aqueous extract (40). Third aqueous extract 40 exits the bottom of third liquid extractor 36 and is purged from the process.

Alternatively, if desired, third aqueous extract 40 can be back extracted with an organic solvent, such as the first or second organic solvent, for example ethyl acetate, before being purged from the process. The recycle stream from this back extraction of third aqueous extract 40 can be combined with stream 42 (the solvent feed to extractor 36) or the recycle stream can be fed to second liquid extractor 22.

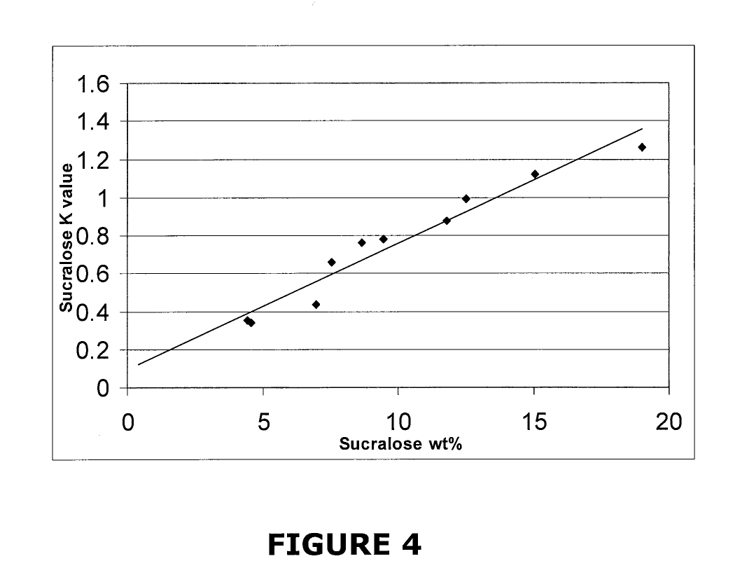

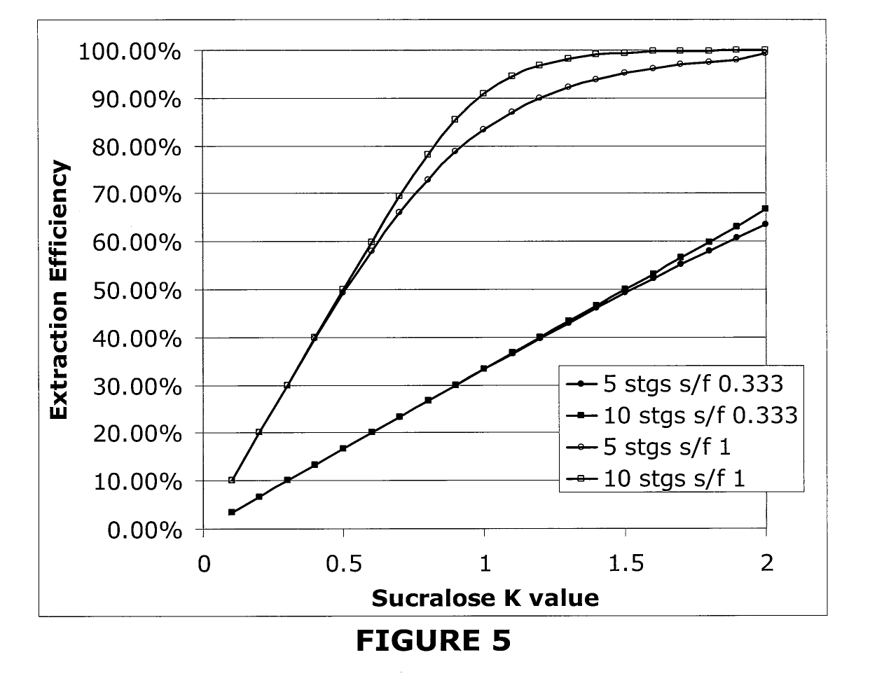

Surprisingly, K, the partition coefficient for sucralose between an organic solvent and water when equal volumes of organic solvent and water are used, is dependent on the concentration of carbohydrates. As shown in FIG. 3, when equal volumes of ethyl acetate and water are used, K increases from 0.4, when the initial concentration of sucralose in the aqueous phase is about 5 wt % to about 1.1 when the initial concentration of sucralose in the aqueous phase is about 15 wt % and about 1.2 when the initial concentration of sucralose in the aqueous phase is about 16 wt %. As shown in FIG. 4, when the ratio of organic solvent to water is 1, the extraction efficiency increases from about 40% to about 90% when the K value increases from about 0.4 to about 1.1 and the extraction efficiency increases from about 40% to over 90% when the K value increases from about 0.4 to about 1.2. Therefore, before the sucralose is extracted into an organic solvent, it is advantageous that sucralose containing aqueous feed stream 18 be concentrated before extraction with the organic solvent. Preferably, the extraction can be carried out under conditions at which the partition coefficient for sucralose between the organic extract and the aqueous extract is at least about 1.0, more preferably at least about 1.1 or at least about 1.2. The partition coefficient is typically in the range of about 1.0 to about 1.6, about 1.1 to about 1.6, about 1.2 to about 1.6, or about 1.25 to 1.6.

Alternatively, the feed stream to concentrator 32 can comprise a sucralose-6-ester, such as sucralose-6-acetate or sucralose-6-benzoate, in addition to, or in place of sucralose, at concentrations the same as those for sucralose, given above. Concentration of a feed stream comprising a sucralose-6-ester also increases the extraction efficiency of the ester into the organic solvent. The concentrator typically increases the concentration of carbohydrates in a sucralose-6-ester containing aqueous feed stream by a factor of about 1.2 to about 4, more typically about 1.5 to about 3. The partition coefficient for a sucralose-6-ester, such as sucralose-6-acetate, between an organic solvent, such as ethyl acetate, and water when equal volumes of organic solvent and water are used is larger than the corresponding value for sucralose measured under the same conditions.

Feed stream 18, or, if concentrator 32 is present, concentrated aqueous stream 34 is fed to the top of a third liquid extractor (36) and a stream of second organic solvent 42, for example a stream of ethyl acetate which if desired may be saturated with water, is fed to the bottom of extractor 36. The mass ratio of organic solvent 42 to aqueous feed stream 34 is in the range of about 1.5 to about 4.0, for example about 1.5 to about 2.0, or about 2.0 to about 2.5, or about 2.5 to about 4.0. Six to twelve extraction stages can be used. However, if the number of theoretical stages of extraction in third liquid extractor 36 were increased, the amount of organic solvent 42, and consequently the ratio of organic solvent 42 to aqueous feed stream 34, could be reduced.

Any of the organic solvents used as the first organic solvent can be used as the second organic solvent. However, as a result of this extraction step, sucralose is transferred from an aqueous extract to an organic extract, so, if sucralose is to be crystallized from the organic solvent, it is convenient to use a second organic solvent that can be used as the crystallization solvent for sucralose. It is also convenient for the first organic solvent and the second organic solvent to be the same organic solvent. A preferred second organic solvent is ethyl acetate.

Sucralose can be isolated by crystallization from a sucralose containing organic feed stream. However, it has been discovered that the purity of the feed to the crystallization step affects sucralose yield. Lower feed purity produces a lower yield and ultimately lower overall plant yield because a larger amount of sucralose is removed with impurities in the mother liquor.

Referring to FIG. 3, third organic extract 38, which contains sucralose, exits the top of third liquid extractor 36 and is fed to fourth liquid extractor 50. This extraction step is referred to as EXT2B and can be either a batch or continuous process. Fourth liquid extractor 50 can be any type of liquid-liquid extractor known in the art, examples of which are listed above. A stream (52) of water, which if desired is saturated with the second organic solvent, for example water saturated with ethyl acetate, is added to extractor 50. Use of a single stage or a multi-stage counter-current liquid-liquid extraction in the back extraction increases the overall yield of the entire isolation and purification process by 1-2%. For example, a ratio (volume to volume) of water to third organic extract 38 of about 0.5 to about 1.0 can be used. This step further reduces the salt content of the organic extract and increases the purity of the feed to the crystallization step. Because fourth aqueous extract 54 contains sucralose as well as salts, a stream (44) of fourth aqueous extract 54 can be recycled to concentrator 32, and/or, if concentrator 32 is not present, to extractor 36 for recovery of additional sucralose. Alternatively, or additionally, a stream (46) fourth aqueous extract 54 can be recycled to aqueous feed stream 10 for recovery of additional sucralose.

Fourth organic extract 56 is fed to a first crystallizer (58). Crystallization produces a first sucralose product (60) and a first mother liquor (62). This step can be either a batch or a continuous process. First crystallizer 58 can be any type of crystallizer known in the art, for example, Swenson-Walker crystallizer, a mixed tank crystallizer, a fluidized bed crystallizer, a draft tube baffle (DTB) crystallizer, a Krystal continuous crystallizer, a forced circulation evaporative crystallizer, an Oslo type or classified-suspension crystallizer, or an induced circulation crystallizer. In first crystallizer 58, sucralose is separated from a majority of the trichloro saccharides as well as from other impurities. Because the sucralose has been extracted into the second organic solvent, the crystallization solvent is the second organic solvent, for example, ethyl acetate. Alternatively, if the feed stream is a feed stream that contains a sucralose-6-ester, such as sucralose-6-acetete or sucralose-6-benzoate, crystallization in first crystallizer 58 will produce the sucralose-6-ester and a first mother liquor.

Operation of the crystallizer will be determined by, for example, whether the crystallization process is batch or continuous; the type and design of crystallizer chosen; the properties of the crystallization solvent chosen, including, for example, its boiling point, its heat of vaporization, the solubility of sucralose and/or the sucralose-6-ester as a function of temperature in the chosen solvent, and the solubility of the impurities as a function of temperature in the chosen solvent; the concentration of sucralose and/or the sucralose-6-ester in the feed to the crystallizer; the purity of the feed to the crystallizer; the nature of the impurities in the feed; mixing requirements of the crystallizer; seeding requirements; and solid-liquid separation requirements; as well as the crystal size, crystallization rate, product yield, and product purity desired. The temperature of the solvent in the crystallizer can be controlled by a number of means. A jacketed vessel or a vessel with one or more internal cooling coils can be used. The solution/slurry in the crystallizer can be pumped through an external heat exchanger. Evaporative cooling can be used for temperature control by altering the pressure in the crystallizer, which, in turn, controls the boiling point of the solvent. Too high a temperature can cause product degradation. If the temperature is too low, there may not be enough heat available to evaporate the solvent. Some of the variables that affect the crystallization are the density or specific gravity of the slurry in the crystallizer, mixing intensity, and crystallization rate. In a batch crystallizer, evaporation of the solvent can be used to concentrate the solution, and the sucralose and/or the sucralose-6-ester can be crystallized by cooling the solution. If necessary, crystallization can be induced by, for example, addition of seed crystals. In a continuous crystallizer, such factors as feed rate, slurry density, residence time of the sucralose and/or the sucralose-6-ester in the crystallizer, and manner of product removal from the crystallizer need to be considered.

If the feed stream is a sucralose containing feed stream, crystallization produces a first sucralose product (60) and a first mother liquor (62). First sucralose product 60 can be separated from first mother liquor 62 by any convenient solid-liquid separation technique known in the art, such as filtration, for example, by pressure filtration, rotating filters, continuous rotary vacuum filters, continuous moving bed filters, or batch filters, or by batch or continuous solid-liquid centrifugation. If desired, the first sucralose product can be further purified by additional processing steps. If desired, first mother liquor 62, which contains sucralose in addition to impurities, can be processed further to recover additional sucralose.

If the feed stream is a sucralose-6-ester containing feed stream, the sucralose-6-ester product can also be separated from the mother liquor by any of these processes. If desired, the sucralose-6-ester product can be further purified by additional processing steps. If desired, the mother liquor, which contains sucralose-6-ester in addition to impurities, can be processed further to recover additional sucralose-6-ester. A sucralose-6-ester can be converted to sucralose as described below.

### Preparation of Sucrose-6-Ester

Selective protection of the 6-hydroxyl of sucrose can be carried out by reaction of sucrose with a carboxylic acid anhydride, such as acetic anhydride or benzoic anhydride, in an anhydrous polar aprotic solvent in the presence of an organotin-based acylation promoter, at a temperature and for a period of time sufficient to produce the sucrose-6-ester. The 6-ester group shields the hydroxyl on the 6 position from the chlorination reaction. Accordingly, any ester group that is stable to the conditions of the chlorination reaction and which can be removed under conditions that do not affect the resulting sucralose can be used. When sucrose-6-acetate is prepared, 1,3-diacetoxy-1,1,3,3-tetrabutyldistannoxane, for example, can be used as the organotin-based acylation promoter and acetic anhydride as the carboxylic acid anhydride. Preparation of sucrose-6-esters is disclosed in, for example, O'Brien, U.S. Pat. No. 4,783,526; Navia, U.S. Pat. No. 4,950,746; Simpson, U.S. Pat. No. 4,889,928; Neiditch, U.S. Pat. No. 5,023,329; Walkup, U.S. Pat. No. 5,089,608; Vernon, U.S. Pat. No. 5,034,551; Sankey, U.S. Pat. No. 5,470,969; Kahn, U.S. Pat. No. 5,440,026; Clark, U.S. Pat. No. 6,939,962, and Li, U.S. Pat. Pub. 2007/0227897 A1; the disclosures of which are all incorporated herein by reference.

### Preparation of Sucralose-6-Ester Containing Feed Streams

To convert sucrose-6-ester to sucralose-6-ester, the hydroxyls at the 4, 1′, and 6′ positions of the sucrose-6-ester are converted to chloro groups, and the stereochemical configuration at the 4 position is inverted. Conversion of the hydroxyls in the 4, 1′, and 6′ positions of the ester to chloro groups with inversion of the stereochemical configuration at the 4 position is disclosed in Walkup, U.S. Pat. No. 4,980,463; Jai, U.S. Pat. Pub. 2006/0205936 A1; and Fry, U.S. Pat. Pub. 2007/0100139 A1; the disclosures of which are all incorporated herein by reference.

The chlorination process comprises the following steps. A reaction mixture is prepared comprising the sucrose-6-ester, a tertiary amide, and at least seven molar equivalents of a chlorination agent. For example, in one process, the sucrose-6-ester can be added in a feed stream that comprises about 20 wt % to about 40 wt % of the sucrose-6-ester. The ratio by weight of tertiary amide to total carbohydrate in the reaction mixture may be about 5:1 to about 12:1. Alternatively, a preformed chloroformiminium salt, such as (chloromethylene)dimethylammonium chloride (Arnold's reagent), can be used. (Chloromethylene)dimethylammonium chloride can be prepared, for example, by the reaction of phosgene with N,N-dimethyl formamide. Typically, the molar ratio of the (chloromethylene)dimethylammonium salt to the sucrose-6-ester is about 7:1 to about 11:1.

Subsequently, the hydroxyl groups of the sucrose-6-ester converting the hydroxyls at the 2, 3, 4, 1′, 3′, 4′, and 6′ positions are converted to O-alkylformiminium groups. The resulting reaction mixture is heated at a temperature or temperatures and for a period of time or times sufficient to produce a product containing a derivative of sucralose-6-ester in which the remaining hydroxyl groups remain as O-alkylformiminium groups. For example, Walkup, U.S. Pat. No. 4,980,463, the disclosure of which is incorporated herein by reference, and Fry, U.S. 2007/0100139, the disclosure of which is incorporated herein by reference, disclose such processes.

Because formation of a chloroformiminium salt or Vilsmeier reagent is not essential to the chlorination reaction, chlorination agent refers to any compound that can be used to form a chloroformiminium salt or Vilsmeier reagent, or that can convert the hydroxyl groups of a sucrose-6-ester to chloro groups. Some chlorination agents that can be used include, for example, phosgene, phosphorus oxychloride, phosphorus pentachloride, thionyl chloride, sulfuryl chloride, oxalyl chloride, trichloromethyl chloroformate (“diphosgene”), bis(trichloromethyl) carbonate (“triphosgene”), and methane sulfonylchloride. Tertiary amides that can be used include, for example, N,N-dimethyl formamide (DMF), N-formyl piperidine, N-formyl morpholine, and N,N-diethyl formamide. When N,N-dimethyl formamide is used as the tertiary amide, it can also be used as the reaction solvent. Co-solvents can be used at up to about 80 vol % or more of the liquid phase of the reaction medium. Useful co-solvents are those which are both chemically inert and which provide sufficient solvent power to enable the reaction to become essentially homogeneous at the monochlorination stage, for example toluene, o-xylene, 1,1,2-trichloroethane, 1,2-diethoxyethane, and diethylene glycol dimethyl ether.

Quenching of the reaction mixture restores the hydroxyl groups at the 2, 3, 3′, and 4′ positions and forms the sucralose-6-ester. The reaction mixture can be quenched by the addition of about 0.5 to about 2.0 molar equivalents, typically about 1.0 to about 1.5 molar equivalents, of alkali relative to the amount of chlorination agent used in the reaction. An aqueous solution of an alkali metal hydroxide, such as sodium or potassium hydroxide; an aqueous slurry of an alkaline earth metal hydroxide, such as calcium hydroxide; or aqueous ammonium hydroxide can be used to quench the reaction. For example, an aqueous solution of an alkali metal hydroxide, such as aqueous sodium hydroxide, that contains about 5 wt % to about 35 wt %, typically about 8 wt % to about 20 wt %, and preferably about 10 wt % to about 12 wt % can be used.

As described below, quenching can be carried out by addition of alkali to the reaction mixture, by the dual stream process, or by the circulated process. In each case pH and temperature are controlled during addition of the alkali. Quenching is typically carried out at a pH between about 8.5 to about 10.5 and at a temperature of about 0° C. to about 60° C. Preferably, the pH should not be permitted to rise above about 10.5 during the course of the quenching reaction.

In the dual stream process, quenching is carried out by slow addition of the aqueous alkali with simultaneous slow addition of the chlorination reaction material into a reaction vessel. The chlorination reaction mixture and aqueous alkali are simultaneously added slowly until the desired quantity of chlorination reaction mixture has been added. Further aqueous alkali is added until the desired pH is reached. Then the temperature and pH are maintained at the desired levels for the remainder of the reaction. This process can be a batch or continuous process.

In the circulated process, quenching is carried out by circulating the chlorination reaction mixture from a vessel through a circulation loop. Chlorination reaction mixture and aqueous alkali are added slowly into this circulation loop. Sufficient aqueous alkali is added until the desired pH is reached. Then the temperature and pH are maintained at the desired levels for the remainder of the reaction. This process can be a batch or continuous process.

Following quenching, the reaction mixture can be neutralized by the addition of aqueous acid, for example aqueous hydrochloric acid. The resulting mixture comprises sucralose 6-ester, other carbohydrate including chlorinated carbohydrate impurities, unreacted tertiary amide, and salts in an aqueous solvent in which the predominant solvent is water.

This mixture can be concentrated and used as the sucralose 6-ester containing aqueous feed stream for the process in which the sucralose is purified at the ester stage. After purification, the resulting purified sucralose 6-ester is deacetylated to sucralose, and the sucralose crystallized. Because the sucralose 6-ester is less polar than sucralose, the partition coefficients for the sucralose 6-ester between an organic solvent and water are much higher than the partition coefficients for the sucralose between an organic solvent and water. Consequently, the sucralose 6-ester is efficiently extracted into the organic solvent rather than remaining in the aqueous solution.

Alternatively, the sucralose 6-ester containing aqueous feed stream can be used in a process, described below, in which the sucralose 6-ester is converted to sucralose before purification.

### Conversion of Sucralose-6-Ester to Sucralose

The sucralose 6-ester containing aqueous feed stream typically comprises both sucralose and sucralose-6-ester. Methods for hydrolyzing sucralose-6-ester are disclosed, for example in Catani, U.S. Pat. Nos. 5,977,349, 6,943,248, 6,998,480, and 7,049,435; Vernon, U.S. Pat. No. 6,890,581; El Kabbani, U.S. Pat. Nos. 6,809,198, and 6,646,121; Navia, U.S. Pat. Nos. 5,298,611 and 5,498,709, and U.S. Pat. Pub. 2004/0030124; Liesen, U.S. Pat. Pub. 2006/0188629 A1; Fry, U.S. Pat. Pub. 2006/0276639 A1; El Kabbani, U.S. Pat. Pub. 2007/0015916 A1; Deshpande, U.S. Pat. Pub. 2007/0160732 A1; and Ratnam, U.S. Pat. Pub. 2007/0270583 A1; the disclosures of which are all incorporated herein by reference.

For example, (a) sucralose-6-ester can be hydrolyzed to sucralose by raising the pH of the reaction mixture to about 11±1 at a temperature and for a time sufficient to effect removal of the protecting group, and (b) the tertiary amide is removed by, for example, stream stripping. Either step (a) or step (b) can be carried first. Alternatively, conversion of sucralose-6-ester to sucralose can be carried in methanol containing sodium methoxide. A trans-esterification reaction occurs that forms sucralose and the methyl ester of the acid, for example methyl acetate when the sucralose-6-ester is sucralose-6-acetate. The methyl ester of the acid can be removed by distillation, and the resulting sucralose containing product dissolved in water.

### INDUSTRIAL APPLICABILITY

The process of the invention is useful in the preparation and purification of sucralose and sucralose-6-esters, such as sucralose-6-acetate. In one aspect, the invention provides an increased yield of crystalline sucralose from a feed of an impure aqueous sucralose solution such as one obtained by alkaline deacylation of a 6-O-acyl sucralose precursor and followed by neutralization.

Sucralose is a high-intensity sweetener that can be used in many food and beverage applications, as well as in other applications. Such applications include, for example, beverages, combination sweeteners, consumer products, sweetener products, tablet cores (Luber, U.S. Pat. No. 6,277,409), pharmaceutical compositions (Luber, U.S. Pat. No. 6,258,381; Roche, U.S. Pat. No. 5,817,340; and McNally, U.S. Pat. No. 5,593,696), rapidly absorbed liquid compositions (Gelotte, U.S. Pat. No. 6,211,246), stable foam compositions (Gowan, Jr., U.S. Pat. No. 6,090,401), dental floss (Ochs, U.S. Pat. No. 6,080,481), rapidly disintegrating pharmaceutical dosage forms (Gowan, Jr., U.S. Pat. No. 5,876,759), beverage concentrates for medicinal purposes (Shah, U.S. Pat. No. 5,674,522), aqueous pharmaceutical suspensions (Ratnaraj, U.S. Pat. No. 5,658,919; Gowan, Jr. U.S. Pat. Nos. 5,621,005 and 5,374,659; and Blase, U.S. Pat. Nos. 5,409,907 and 5,272,137), fruit spreads (Antenucci, U.S. Pat. No. 5,397,588; and Sharp, U.S. Pat. No. 5,270,071), liquid concentrate compositions (Antenucci, U.S. Pat. No. 5,384,311), and stabilized sorbic acid solutions (Merciadez, U.S. Pat. No. 5,354,902). The determination of an acceptable sweetness can be accomplished by a variety of standard “taste test” protocols known in the art which are well known to those skilled in the art, such as, for example, the protocols referred to in Merkel, U.S. Pat. No. 6,998,144, and Shamil, U.S. Pat. No. 6,265,012.

The advantageous properties of this invention can be observed by reference to the following examples which illustrate but do not limit the invention.

### EXAMPLES

### Example 1

This example was generated using a mathematical model that included both a first extraction process (EXT1), a back extraction (EXT1B) of the first organic extract (16), and recycle of the second aqueous extract (12) to the first extraction process. The calculations used in the model were derived from theoretical equations fitted to actual pilot plant data. FIG. 1 shows a flow diagram of the modeled process.

FIG. 2 shows the results from multiple model runs in which the mass ratio of first organic solvent 14 to the combined aqueous feed stream in the first extraction was varied. The number of separation stages in the back extraction was adjusted to maintain an equivalent overall extraction yield. The amount of sucralose extracted into the first organic extract 16 during the first extraction step is shown on the left hand axis. The purity of the sucralose produced by the process is shown on the right hand axis.

As can be seen from FIG. 2, when the mass ratio of organic solvent 14 to combined aqueous feed stream in the first extraction step is about 0.4 or greater, about 50% or more of the sucralose is extracted into first organic extract 16. When the mass ratio is 0.6 or greater, greater than about 65% of the sucralose is extracted into first organic extract 16. Surprisingly, the level of impurities in first aqueous extract 18 is reduced significantly, with little or no reduction in overall sucralose yield, when higher organic solvent to the combined aqueous feed stream ratios are used in the first extraction step. As can also be seen from FIG. 2, product purity begins to level out near 75%, when nearly 90% of the sucralose is extracted into first organic extract 16.

### Example 2

This example shows the effect of sucralose concentration on the partition coefficient of sucralose between an organic phase and aqueous phase. Aqueous solutions of sucralose were prepared at various carbohydrate concentrations. An equal volume of ethyl acetate was then added to each solution and the two phases mixed thoroughly. After the two phases separated, the carbohydrate concentration in each phase was determined. The K value was calculated by dividing the concentration of sucralose in the ethyl acetate phase by the concentration of sucralose in the aqueous phase. FIG. 4 shows the effect of carbohydrate concentration on the partition coefficient, K. The larger the K value the more readily sucralose is extracted into the ethyl acetate phase.

### Example 3

This example measures the effect of concentration on sucralose yield. Two different organic solvent to sucralose containing aqueous feed stream ratios (volume to volume) were used: about 3.7:1 and about 3.0:1. The organic solvent was ethyl acetate. The results are shown in Table 1. In Table 1, “Solvent:Feed” is the ratio (volume to volume) of the organic solvent to the sucralose containing aqueous feed stream. “Carbohydrates” is the wt % of carbohydrates in the sucralose containing aqueous feed stream. “Salt” is the wt % of salt in the sucralose containing aqueous feed stream. “Yield” is the percent of sucralose recovered from the organic phase of the extraction. Because multi-stage contacting devices were used for this testing, K values could not be determined directly from this data and compared to the values determined in Example 2.

TABLE 1

| Concentration | Solvent:Feed | Sucralose (wt %) | Salt (%) | Yield (%) |
|---|---|---|---|---|
| Low | 3.68 | 6.97 | 8.37 | 97.54 |
| Low | 2.95 | 6.17 | 7.78 | 97.31 |
| High | 3.69 | 13.77 | 15.00 | 99.99 |
| High | 3.91 | 14.46 | 15.13 | 99.94 |
| High | 3.76 | 14.69 | 14.98 | 99.98 |
| High | 3.18 | 13.82 | 14.95 | 100 |

These experiments show a dramatic increase in extraction efficiency when the sucralose containing aqueous feed stream is more concentrated. These extraction efficiencies were inserted into a mathematical model of a purification process for sucralose. Increasing the EXT2 extraction efficiency from 97.5% to 99.5% improves the overall yield of sucralose by >5.5%, when using the same solvent to feed ratio and number of extraction stages.

### Example 4

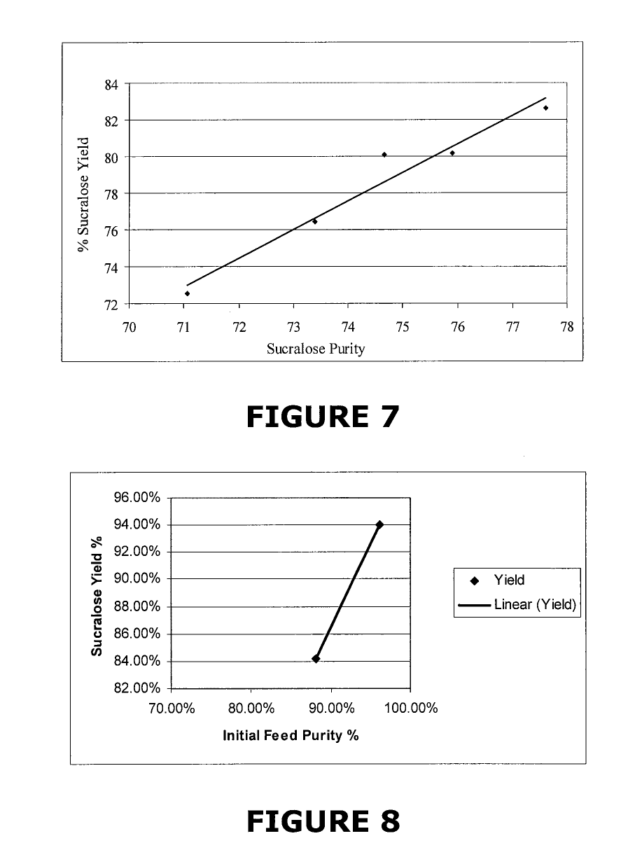

This example shows the influence of feed purity on crystallizer yield. Six different feed solutions of varying feed purity were prepared. Each solution was loaded into a rotary evaporator and heated to a pre-set temperature to make sure all the carbohydrates were completely in solution. Each solution was then cooled to 40° C., and a small amount of sucralose seed crystals were added to each solution. Each solution was then allowed to crystallize for 18 hr. The precipitates were separated from the mother liquor, and a material balance was completed to determine the sucralose yield. The results are shown in FIG. 7. The plot of yield of sucralose against initial purity has a slope of 1.55, indicating that increasing the feed purity by 2% produces an increase in crystallizer yield of just over 3%.

### Example 5

This example shows the influence of feed purity on crystallizer yield. The procedure of Example 4 was repeated except that two feed solutions of higher purity were used. The results are shown in FIG. 8. This example is similar to Example 4 except that the feed purity for the 2 runs was significantly higher. This experiment indicates that the crystallizer yield increases by a factor of 1.25 for every 1% increase in feed purity.

### Example 6

This example is a mathematical model of the process that determines the influence of solvent to feed ratio and the number of stages in the EXT2B extraction on overall yield of the purification and isolation process. The model is an iterative spreadsheet that links all purification techniques and recycle streams together. The data from Example 4 was used as the basis for the model work presented here.

A base case model was run to determine the purity of the feed to the crystallizer without a back extraction (step EXT2B). Keeping all other parameters constant, two variables were altered to determine the optimal conditions for the extraction: the ratio of water (52) to third organic extract 38 (“EXT2B S:F Ratio”) (volume to volume) and the number of extraction stages (“EXT2B Stages”) in the fourth liquid extraction (step EXT2B). The ratio (“EXT2 S:F Ratio”) of second organic solvent 42 to concentrated feed stream 34 in the third liquid extraction (EXT2) was not varied. “Purity Increase” refers to increase in purity of fourth liquid extract 38. “P1 Increase” refers to the increase in yield in the first crystallization step. “Overall Yield Increase” refers to the yield increase for the entire purification and recovery process. Purity increase was calculated against the base case and this purity multiplied by the crystallization yield factor determined from the data included in Example 4. The projected yield increase was then inserted into the first crystallization part of the spreadsheet and the calculation iterated to steady state. The overall purification area yield produced for each case was then compared against the base case yield to determine the overall yield increase. The results are shown in Table 2.

TABLE 2

| | | EXT2B | EXT2 | | | Overall |
|---|---|---|---|---|---|---|
| | EXT2B | S:F | S:F | Purity | P1 Yield | Yield |
| | Stages | Ratio | Ratio | increase | Increase | Increase |
| Base Case | 0^{a} | 0.00^{a} | 3.0 | 0.00% | 0.00% | 0.00% |
| 1 | 1 | 0.10 | 3.0 | 0.37% | 0.56% | 0.12% |
| 2 | 12 | 0.10 | 3.0 | 0.61% | 0.91% | 0.23% |
| 3 | 12 | 0.30 | 3.0 | 1.36% | 2.04% | 0.52% |
| 4 | 12 | 0.50 | 3.0 | 1.81% | 2.72% | 0.66% |
| 5 | 12 | 0.75 | 3.0 | 2.23% | 3.35% | 0.78% |
| 6 | 6 | 0.75 | 3.0 | 2.20% | 3.30% | 0.77% |
| 7 | 6 | 0.50 | 3.0 | 1.79% | 2.68% | 0.65% |
| 8 | 3 | 0.75 | 3.0 | 2.04% | 3.06% | 0.70% |
| 9 | 1 | 0.75 | 3.0 | 1.36% | 2.04% | 0.44% |
| 10 | 12 | 1.00 | 3.0 | 2.56% | 3.85% | 0.87% |
| 11 | 12 | 1.30 | 3.0 | 2.88% | 4.33% | 0.95% |

^{a}EXT2B step omitted.

The disclosure of the invention includes the following claims. Having described the invention, we now claim the following and their equivalents.

What is claimed is:

1. A process comprising, in order, the steps of:
a) providing an aqueous feed stream comprising chlorinated saccharide impurities and a carbohydrate selected from the group consisting of sucralose, sucralose-6-esters, and mixtures thereof;
b) optionally, concentrating the aqueous feed stream;
c) extracting the aqueous feed stream with an organic solvent and producing a first organic extract and a first aqueous extract, in which the organic solvent is immiscible with water, and in which the carbohydrate is preferentially extracted into the first organic extract;
d) extracting the first organic extract with water and producing a second organic extract and a second aqueous extract, in which the carbohydrate preferentially remains in the second organic extract; and
e) crystallizing the carbohydrate from the second organic extract;
wherein the process further comprises recycling the second aqueous extract to the aqueous feed stream.

2. The process of claim 1 in which step b) is present and in which in step b) the concentration of carbohydrates in the aqueous feed stream is increased by a factor of about 1.1 to about 4.0.

3. The process of claim 1 in which the ratio of water to first organic extract in step d) is about 0.5 to about 1.0, volume to volume, and the number of extraction stages in step c) is 6 to 12.

4. The process of claim 1 in which the carbohydrate is a sucralose-6-ester.

5. The process of claim 1 in which the carbohydrate is sucralose-6-acetate.

6. The process of claim 4, further comprising, after step e), deacylating the carbohydrate to form sucralose and subsequently purifying and isolating the sucralose.

7. The process of claim 1 in which the carbohydrate is sucralose.

8. The process of claim 7, additionally comprising, after step e), the step or steps of purifying and isolating the sucralose.

9. The process of claim 1 in which the organic solvent is ethyl acetate.

10. A process comprising the steps of:
a) extracting an aqueous feed stream comprising sucralose, salts and chlorinated saccharide impurities with a first organic solvent and producing a first organic extract and a first aqueous extract, in which the first organic solvent is immiscible with water, and in which a portion of the sucralose passes into the first organic extract;
b) optionally, extracting the first organic extract with an aqueous solvent to produce a second organic extract and a second aqueous extract, in which the sucralose preferentially passes into the second aqueous extract, and recycling the second aqueous extract to step a);
c) optionally, concentrating the first aqueous extract;
d) extracting the first aqueous extract with a second organic solvent and producing a third organic extract and a third aqueous extract;
e) extracting the third organic extract with water and producing a fourth organic extract and a fourth aqueous extract; and
f) crystallizing the sucralose from the fourth organic extract.

11. The process of claim 10, additionally comprising, after step e), a step of adding the fourth aqueous extract to the first aqueous extract.

12. The process of claim 10 in which the first organic solvent and the second organic solvent are the same.

13. The process of claim 10 in which the first organic solvent is ethyl acetate.

14. The process of claim 10 in which the second organic solvent is ethyl acetate.

15. The process of claim 10 in which the chlorinated impurities comprise tetrachloro saccharides, in step a) greater than 50% of the sucralose and at least 95% of the tetrachloro saccharides in the aqueous feed stream pass into the first organic extract, and in step b) greater than 90% of the sucralose in the first organic extract passes into the second aqueous extract.

16. The process of claim 10 in which step c) is present and in which in step c) the concentration of carbohydrates in the first aqueous extract is increased by a factor of about 1.1 to about 4.0.

17. The process of claim 10 in which the ratio of water to third organic extract in step e) is about 0.5 to about 1.0, volume to volume, and the number of extraction stages in step d) is 6 to 12.

18. The process of claim 10, additionally comprising, after step f), the step or steps of purifying and isolating the sucralose.
